(19) United States
(12) Patent Application Publication
(10) Pub. No.: US 2004/0132204 A1
(43) Pub. Date: Jul. 8, 2004

(54) Portable pH detector

(76) Inventors: Jung-Chuan Chou, Yunlin (TW); Lan Pin Liao, Taipei (TW); Chan Hai Wei, Ilan (TW)

Correspondence Address: BIRCH STEWART KOLASCH & BIRCH, PO BOX 747, FALLS CHURCH, VA, 22040-0747, US

(21) Appl. No.: 10/335,850

(22) Filed: Jan. 3, 2003

(51) Int. Cl. G01N 33/00

(52) U.S. Cl. 436/163; 422/68.1

(57) ABSTRACT

A portable pH detector. A pH-ISFET is immersed into a solution, and the gate voltage of the pH-ISFET is detected by a signal detection unit. The level of the detected voltage is adjusted by a gain control and level shift unit and is converted by an A/D converter circuit and then output to a 8051 microcontroller to compute the pH of the solution, with the computed result displayed on a LCD.

### BACKGROUND OF THE INVENTION

1. Field of the Invention

The invention relates to a pH detector, and more particularly to a portable pH detector compatible with several types of ion sensitive field effect transistors (ISFET) to detect and display pH of a solution.

2. Description of the Related Art

ISFET (Ion Sensitive Field Effect Transistor) is constructed by substituting a sensing film for the metal gate on the gate oxide of a tradition MOSFET (Metal-Oxide-Semiconductor Filed Effect Transistor). When the ISFET is dipped into a solution, the interfacial potential between the sensing film and the solution will influence the semiconductor surface since only an extremely thin dielectric (that is, the gate oxide) separates the sensing film from the semiconductor surface. This influences the charge density in the inversion layer of the semiconductor surface, and thereby modulates the channel current through the ISFET. Thus, by utilizing this characteristic, the pH or other ion concentration in the solution can be obtained from the measurement of source/drain current and the gate voltage of the ISFET. The potential difference on the interface between the sensing film and the solution is related to the ion activity in the solution. The hydrogen ion activity in the solution can be measured using different channel currents caused by different interfacial potential differences in various solutions with different hydrogen ion activity.

Patents related to the formation of the ISFET or measurement thereof are listed hereinafter.

U.S. Pat. No. 4,691,167 discloses a method of measuring ion activation in a solution by combining the ISFET, the reference electrode, the temperature sensor, amplifier circuit and a calculation and memory circuit. Since the sensitivity is a function of the temperature and drain current of ISFET and is decided by a variable of gate voltage, the sensitivity can be obtained by calculating formulas stored in memory.

U.S. Pat. No. 5,309,085 integrates the measurement circuit of a creature sensor having ISFET on a wafer. The measure circuit has two ISFET devices, an enzyme ISFET and a reference electrode FET, whose output signals can be amplified by a differential amplifier.

U.S. Pat. No. 5,911,873 discloses a method and apparatus for determining and controlling the zero potential point by measuring currents and gate voltages. Also, an apparatus for measuring the ion concentration in the solution is provided, with an ISFET, a reference electrode, a control circuit, a memory, a measuring circuit and a diagnostic circuit. The control circuit operates the ISFET under a drain-source current and plurality of continuous drain currents and gate voltages. The measuring circuit and diagnostic circuit measure the ion concentration and the device characteristics. Finally, the measuring results are recorded.

U.S. Pat. No. 4,384,925 discloses an apparatus for sensing gases in the environment, wherein electrochemical sensing procedures not only monitor ambient continuously for the presence of such gases, but also periodically and automatically calibrate and adjust the sensing instrument to accommodate changing conditions over time. The instrument is connected to a microprocessor, and is adjusted to zero to accommodate drift during the recalibration procedure.

U.S. Pat. No. 4,641,084 discloses an apparatus for measuring a concentration of a specific ion contained in a liquid. The apparatus includes a reference electrode, an ISFET, a series circuit of reference resistor, a constant voltage supply connected across drain and source of the ISFET and a potential control circuit having inputs connected across the reference resistor to detect a potential difference across the reference resistor to control a source or drain potential of the ISFET. In this way, the potential difference remains at a predetermined value and the source or drain potential is measured as a measure of the ion concentration by a voltmeter.

U.S. Pat. No. 4,397,714 discloses a solid state chemically sensitive integrated circuit including three FETs fabricated in a single semiconductor substrate. The gate of a first FET is overlaid with a chemically sensitive element adapted to create an electrochemical potential at the gate when exposed to selected chemical substances. A second and third FET are used as switches to isolate the gate of the first FET from external signals. The presence of the second and third FETs allows the first FET to be protected from static shock during routine handing and when the device is not in use.

U.S. Pat. No. 3,684,159 discloses a completely portable, self-powered system for rapidly and accurately measuring whole blood hematocrit and pH and levels of sodium, potassium, chloride and the other ions in blood and other fluids. The system induces individual selective ion electrodes, a reference electrode, a high-impedance solid- state electrometer, a high-impedance electrode switch, and an integral rechargeable power supply.

U.S. Pat. No. 4,207,159 discloses an apparatus for measuring oxygen concentration, wherein the apparatus includes a probe with an oxygen ion conductive solid electrolyte layer, potentiometer and a DC power source connected to the probe. During measurement, the DC power source keeps a current flowing through the electrolyte layer between the two electrode layers to maintain a reference oxygen partial pressure at the interface between the electrolyte layer and the reference electrode layer.

U.S. Pat. No. 4,532,013 discloses a method for calibrating sensor drift over time. The sensor is exposed to ambient air forming a standard gas and the output thereof compared with a standard value in the circuit therewith or recalled from a memory. By changes in applied voltage to the sensor and tests for current therethrough, the current generates an actual operating curve which can be compared with stored test curves from which, upon extrapolation, a tendency to failure can be predicted and recognized.

However, potential hydrogen ion detectors are usually applied for only one single sensing element, and cannot apply to the pH-ISFETs fabricated in most laboratories for measurement of industrial or domestic sewage.

### SUMMARY OF THE INVENTION

The present invention is directed to a portable pH detector compatible with several types of ion sensitive field effect transistors (ISFET) to detect and display pH of a solution.

In the present invention, a portable pH detector has an ISFET for immersion in a solution, a signal detection unit to supply a constant voltage, a drain and source of the ISFET and a constant current flowing between the drain and the source of the ISFET to detect the gate voltage of the ISFET as an output signal, a gain control and level shift unit to adjust potential of the output signal as a first signal, an A/D converter to convert the first signal into a digital signal, and a microprocessor coupled to the A/D converter to compare the digital signal with a relational table to obtain the pH of the solution.

### DESCRIPTION OF THE DRAWINGS

For a better understanding of the present invention, reference is made to a detailed description to be read in conjunction with the accompanying drawings, in which:

FIG. 1 is a diagram of the pH detector according to the present invention;

FIG. 2 is a structure diagram of the pH-ISFET of the present invention;

FIG. 3 shows the gate voltage of an a-Si:H gate pH-ISFET whose constant drain-source voltage is 0.2 volts and constant drain-source current is 80 μA in various solutions with different hydrogen ion activity;

FIG. 4 is a circuit diagram of the pH-ISFET and the signal detection unit of the present invention;

FIG. 5 is a circuit diagram of the gain control and level shift unit and the A/D converter circuit of the present invention;

FIG. 6 is an operational flowchart of the pH-ISFET according to the present invention.

### DETAILED DESCRIPTION OF THE INVENTION

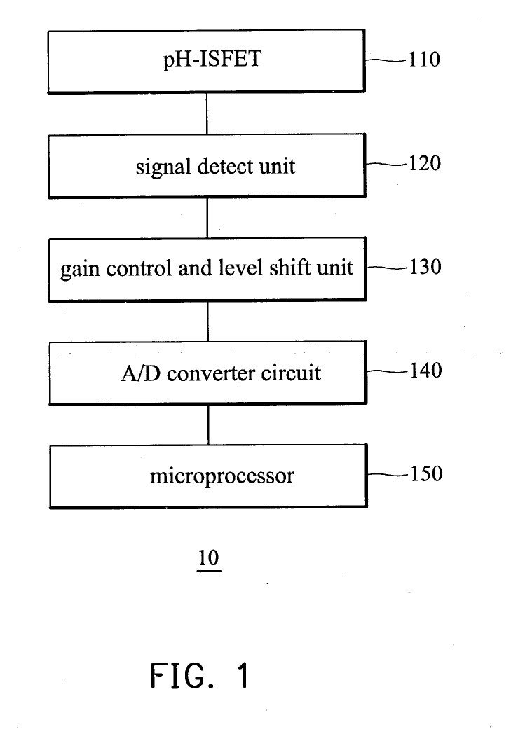

FIG. 1 is a diagram of the portable pH detector according to the present invention. As shown in FIG. 1, the portable pH detector 10 has an ISFET 110, a signal detection unit 120, a gain control and level shift unit 130, an A/D converter 140, and a microprocessor 150. The ISFET 110 has a sensing film for immersion in a solution; the signal detection unit 120 maintains a drain-source voltage and a drain-source current of the ISFET 110 in a constant condition and detects the gate voltage of the ISFET 110 as an output signal. The gain control and level shift unit 130 adjusts level of the output signal as a first signal, and the A/D converter 140 converts the first signal into a first digital signal. The microprocessor 150 is coupled to the A/D converter 140 to compare the first digital signal with a relational table to obtain the pH of the solution.

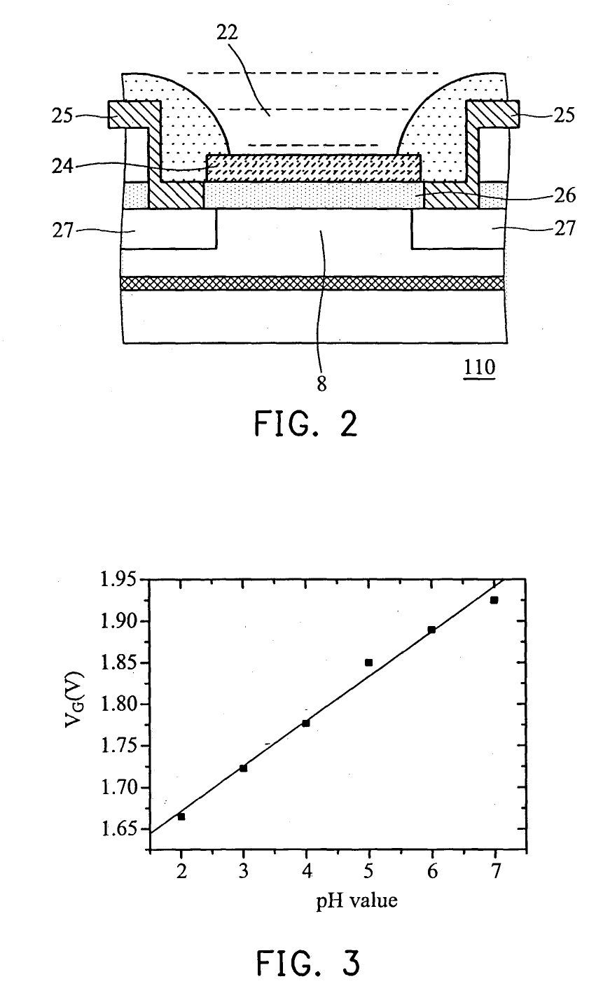

FIG. 2 shows the structure of the pH-ISFET 110. Typically, ISFET (Ion Sensitive Field Effect Transistor) is constructed by substituting a sensing film for the metal gate on the gate oxide of a tradition MOSFET (Metal-Oxide-Semiconductor Field Effect Transistor). When the ISFET is dipped into a solution, the interfacial potential between the sensing film and the solution influences the semiconductor surface since only an extremely thin dielectric (that is, the gate oxide) separates the sensing film from the semiconductor surface. This influences the charge density in the inversion layer of the semiconductor surface, and thereby modulates the channel current through the ISFET. Thus, by utilizing this characteristic, the pH or other ion concentration in the solution can be obtained from the measurement of source/drain current and the gate voltage of the ISFET. The potential difference on the interface between the sensing film and the solution is related to the ion activity in the solution. The hydrogen ion activity in the solution can be measured using different channel currents caused by different interfacial potential differences in various solutions with different hydrogen ion activity.

In the present invention, the sensing film 24, for example, is composed of a-WO₃, a-C:H, a-SnO₂, a-Si:H or the like. In the embodiment of the present invention, the sensing film 24 is composed of a-WO₃, the ISFETs with a sensing film composed of the other material may function as the same as the embodiment of the present invention.

In the present invention, when the ISFET 110 is dipped into a solution 22, the interfacial potential between the sensing film 24 and the solution 26 may influence the semiconductor surface since only an extremely thin dielectric 26(that is, the gate oxide) separates the sensing film 24 and the semiconductor surface. This influences the charge density in the inversion layer of the semiconductor surface, and thereby modulates the channel current through the ISFET 110. Thus, by utilizing this characteristic, the pH or other ion concentration in the solution 22 can be obtained from the measurement of source/drain current I_{DS} and the gate voltage V_{G} of the ISFET 110. The potential difference on the interface between the sensing film 24 and the solution 22 is related to the ion activity in the solution 22. The hydrogen ion activity in the solution 22 can be measured using different channel currents caused by different interfacial potential differences in various solutions with different hydrogen ion activity.

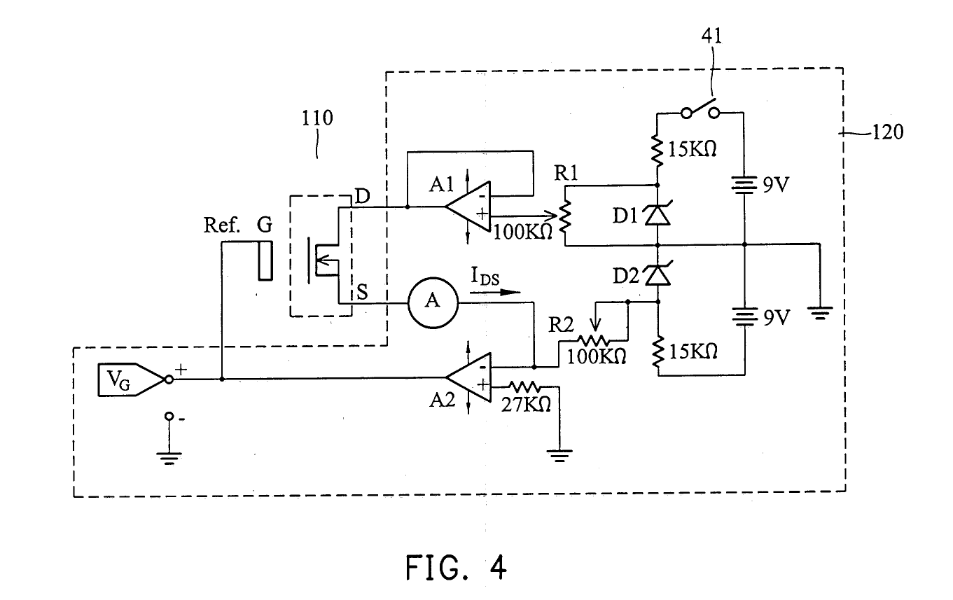

FIG. 3 shows that the relationship between the gate voltage of the pH-ISFET 110 and the pH of the solution under the drain-source voltage of the pH-ISFET 110 is fixed at 0.2 volts and the current between the drain and the source is fixed to 80 μA. As shown in FIG. 3, the sensitivity of the sensing film 24 composed of a-Si:H is 52.92 mV/pH, and other operational parameters and sensitivities of sensing films composed of other materials is shown in Table 1.

TABLE 1

| Sensing film | a-Si:H | a-C:H | a-WO₃ | a-SnO₂ |
|---|---|---|---|---|
| Drain - source | 0.2 | 0.2 | 0.2 | 0.2 |
| voltage | | | | |
| (V) | | | | |
| Drain - source | 80 | 50 | 100 | 50 |
| current | | | | |
| (μA) | | | | |
| Sensitivity | 52.29 | 54.86 | 53 | 57.36 |
| (mV/pH) | | | | |

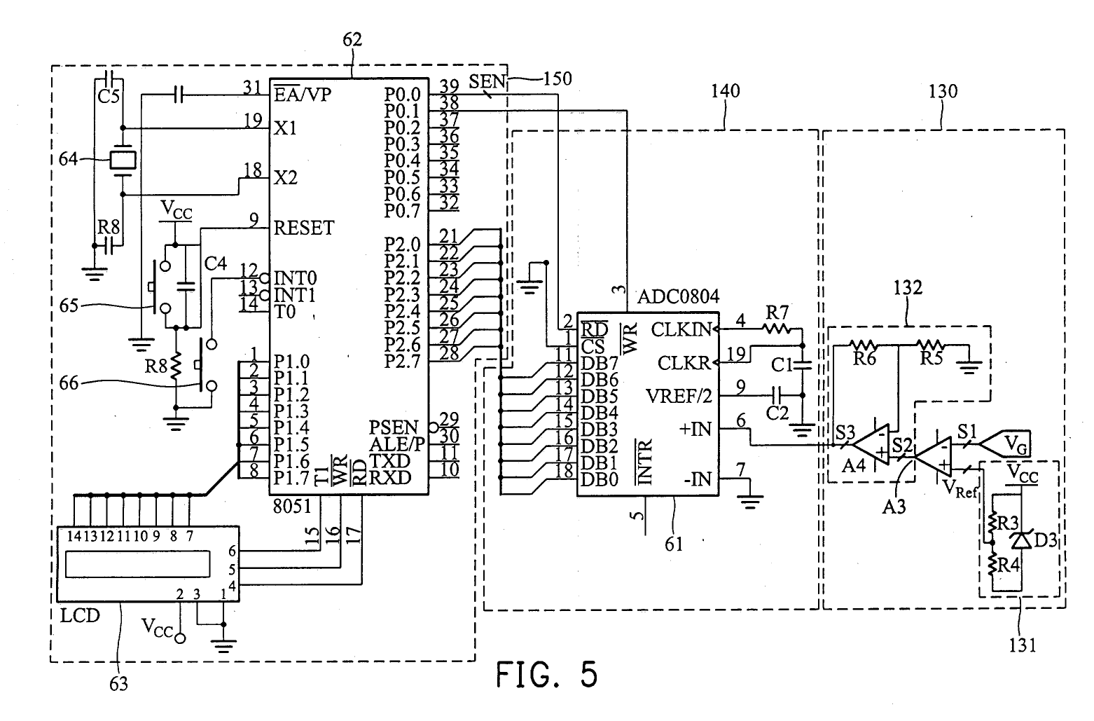

FIG. 4 shows the circuit structure of the signal detect unit 120. The signal detection unit 120 is a constant voltage constant current (CVCC) readout circuit composed of two operational amplifiers A1 and A2 (μA741), two zener diodes D1 and D2, tow resistors R1 and R2, and a power switch 41. As shown in FIG. 4, an operational amplifier A1 is connected to the voltage follower type, and the operational amplifier A2 provides negative feedback to adjust the voltage at the reference electrode to maintain the drain-source current I_{DS} and the drain-source voltage V_{DS} of the ISFET 110 in a constant condition. The signal detect unit 110 maintains the current I_{DS} and the voltage V_{DS} the ISFET 110 in a constant condition due to negative feedback, such that the gate voltage V_{G} of the ISFET 110 varies according to the hydrogen ion activity in the solution 22. The source voltage Vₛ of the ISFET 110 rises as the drain-source current I_{DS} increases due to the negative feedback. The gate voltage V_{G} of the ISFET 110 is pulled down to decrease the drain-source current I_{DS} to maintain the current I_{DS} and the voltage V_{DS} of the ISFET 110 in a constant condition by the negative feedback of the operational amplifier A2. The drain-source voltage V_{DS} may also be adjusted by modifying the resistor R1 and the drain-source current I_{DS} may also be adjusted by modifying the resistor R2. In the present invention, the gate voltage V_{G} of the pH-ISFET 110 measured by the signal detect unit 120 is output as an output signal S1.

FIG. 5 shows circuit structure of the signal detection unit 120, the A/D converter circuit 140 and the microprocessor 150. The gain control and level shift unit 130 includes an instrumentation amplifier A3 (AD620) and an operational amplifier circuit 132. The instrumentation amplifier A3 is coupled to the output signal S1 and a reference voltage V_{Ref} to subtract the level of the reference voltage V_{Ref} from the level of the output signal S1 and then output a subtracted-level signal S2. The operational amplifier circuit 132 includes an operational amplifier A4 (A741) and resistors R5 and R6 coupled to the instrumentation amplifier A3 to amplify the subtracted-level signal S2 for a predetermined time to output as the first signal S3.

The gain control and level shift unit 130 includes a level supply circuit 131 having a first resistor and a second resistor R3 and R4 coupled in series, a zener diode D3 coupled to the first resistor and the second resistor R3 and R4 in parallel, and a DC voltage source Vcc across two ends of the zener diode D3, wherein the DC voltage source Vcc is divided by the first resistor R3 and the second resistor R4 as the reference voltage V_{Ref} for output to the non-inversion input terminal of the instrumentation amplifier A3.

For matching the resolution of the successive A/D converter circuit 140 and the microprocessor 150, the voltage V_{G} detected by the signal detection unit 120 is coupled to the inversion input terminal of the instrumentation amplifier A3. The DC voltage source Vcc is divided by the first resistor R3 and the second resistor R4 as the reference voltage V_{Ref}. In this case, the reference voltage V_{Ref} is 1 volt, and is coupled to the non-inversion input terminal of the instrumentation amplifier A3. Consequently, the instrumentation amplifier A3 subtracts 1 volt (the potential of the reference voltage V_{Ref}) from the voltage V_{G} and outputs a subtracted-level signal S2. The subtracted-potential signal S2 is then amplified 5 times by the amplify circuit 132 composed of the operational amplifier A4 and resistors R5 and R6, and output to the A/D converter circuit 140.

Because the successive 8-bit A/D converter 61 (IC0804) has a lack of resolution and the minimum of the gate voltage V_{G} of the ISFET 110 detected by the signal detect unit 120 is over 1 volt, the present invention subtracts 1 volt from the voltage V_{G} and then amplifies the result to improve resolution. The amplified result may exceed the operational voltage of the A/D converter 61 and cannot work if the 1 volt is not subtracted from the detected voltage V_{G}. Consequently, the present invention subtracts 1 volt from the detected voltage V_{G} and then amplifies the result voltage 5 times to improve resolution.

As shown in FIG. 5, operation of the A/D converter circuit 140 follows. An oscillator composed of resistor R5 and the capacitor C1 is coupled to the 19^{th} pin and the 4^{th} pin of the A/D converter 61, and has a resonance frequency of 600KHz to operate the internal oscillator in the A/D converter 61. Pins /CS and /WR are both set at 1 initially and the /INTR pin is reset to 0, such that the A/D converter circuit is ready for operation. After 100 nanoseconds, the A/D converter 61 begins to convert the output signal S3 (the first signal) output from the operational amplifier A4 into digital data of 8 bits, when one or both of the /CS pin and the /WR pin becomes 1. After conversion, the digital data of 8 bits is stored in the buffer of the A/D converter 61, and the /INTR pin of the A/D converter outputs 0 to indicate that conversion is finished. The three-state buffer of the A/D converter 61 outputs the digital signal of 8 bits through the 11^{th}˜18^{th} pins when both the /CS pin and the /RD pin are “0”.

Further, as shown in FIG. 5, the microprocessor 150 is coupled to the A/D converter circuit 140 and has functions of hardware initialization, two-point self-calibration, data access, data processing, and data delivery/receiving. The microprocessor 150 includes a 8051 microcontroller 62 and a LCD 63, wherein the 8051 microcontroller 62 is coupled to the A/D converter circuit 140, and produces a convert enable signal SEN to the A/D converter 61 to convert the first signal S3 to a digital signal. The microcontroller 62 receives the digital signal for comparison with the relational table to obtain the pH of the solution. The LCD 63 is coupled to the microcontroller 62 to display the pH of the solution.

The relational table is stored in the 8051 microcontroller 62 before testing the solution, and is created by measuring solutions with pH4 and pH7, obtaining two corresponding digital values and by computing the corresponding digital pH values according to the two obtained corresponding digital values.

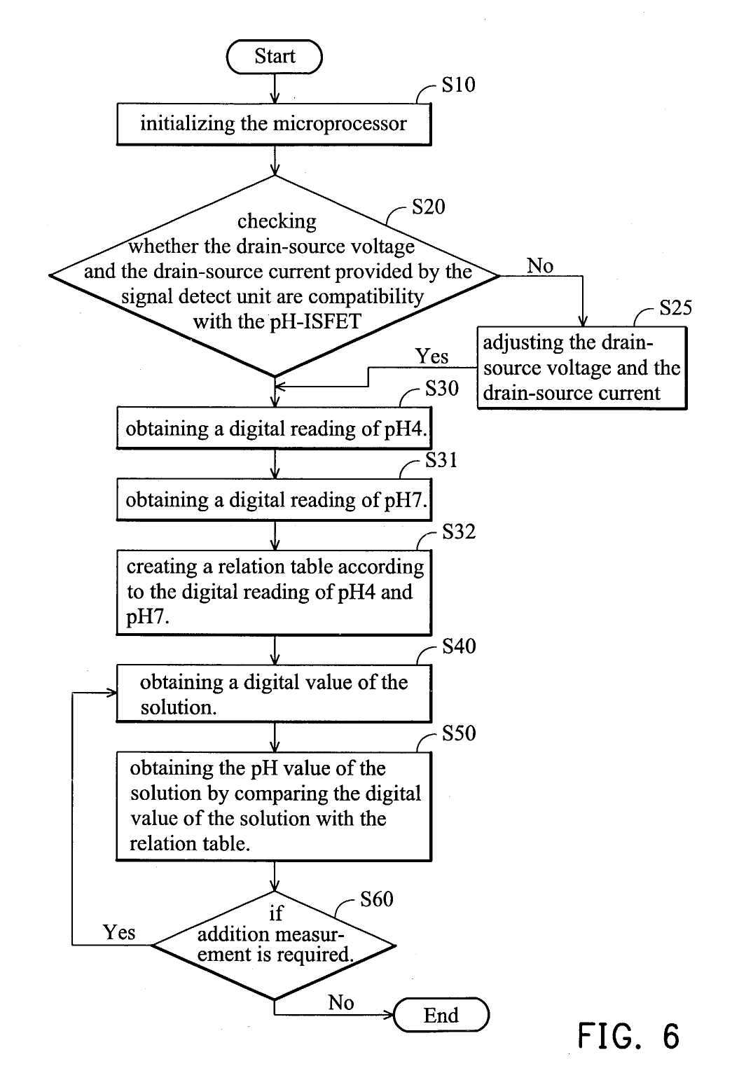

FIG. 6 shows the operational flowchart of the portable pH detector according to the present invention. First, in step S10, the microcontroller 62 and the LCD 63 are initialized when power is turned on. For example, the switch 65 as shown in FIG. 5 is turned on, and the microcontroller 62 and the LCD 63 are initialized to clear data stored in the microcontroller 62 and the LCD 63.

In step S20, the drain-source voltage V_{DS} and the drain-source current I_{DS} provided by the signal detect unit are checked for compatibility with the pH-ISFET. Step S25 is performed when the drain-source voltage and the drain-source current provided by the signal detection unit 120 are not compatible with the pH-ISFET 110. In step S25, the drain-source voltage and the drain-source current are adjusted to provide compatibility with the pH-ISFET 110 by modifying the first resistor R1 and the second resistor R2.

Step S30 is performed to obtain a digital reading of pH4 when the drain-source voltage and the drain-source current provided by the signal detect unit 120 are compatible with the pH-ISFET 110. For example, the LCD 63 may display “PLEASE INPUT pH4 BUFFER”. After 1.5 minutes, a first digital value related to pH4 buffer is obtained for a first reference value of the two-point calibration, through the pH-ISFET 110, signal detection unit 120, gain control and level shift unit 130, and the A/D converter circuit 140.

Step S31 is then performed to obtain a digital reading of pH7 when the drain-source and the drain-source current provided by the signal detection unit 120 are compatible with the pH-ISFET 110. For example, the LCD 63 may display “PLEASE INPUT pH7 BUFFER”. After 1.5 minutes, a second digital value related to pH7 buffer is obtained for a second reference value of the two-point calibration, through the pH-ISFET 110, signal detection unit 120, gain control and level shift unit 130, and the A/D converter circuit 140.

The responding voltage (gate voltage) of the pH-ISFET 100 is not stable when the pH-ISFET 100 is first immersed in the pH4 buffer or the pH7 buffer, such that the present invention waits 1.5 minutes for the responding voltage of the pH-ISFET to stabilize. In the present invention, 1.5 minutes is designated for the output response of most kinds of pH-ISFETs, and it is to be understood that the invention is not limited to the disclosed embodiments.

Next, in step S32, the corresponding digital values of pH1˜pH13 are obtained by the microcontroller 62 when receiving the first reference value with the second reference value, and the digital values are stored into the stack segment of the microcontroller 62 from the higher levels to the grassroots of the relational table of the pH and responding voltage.

Step S40 then is performed to obtain digital data of the solution. For example, the LCD 63 may display “S=*** PLEASE INPUT SOLUTION”, wherein the “***” is the sensitivity value of the pH-ISFET 110. At this time, a measurement related to the pH of the solution is performed after rinsing the pH-ISFET 110 in de-ionized water. The rinsed pH-ISFET 110 is immersed in the solution, and a digital signal related to the pH of the solution is obtained through the pH-ISFET 110, signal detection unit 120, gain control and level shift unit 130, and the A/D converter circuit 140.

The digital signal related to the pH of the solution is output to the microcontroller 62, which then compares the digital signal with the relational table stored in the stacked segment of the microcontroller 62 to obtain the pH of the solution in step S50. The microcontroller 62 outputs the computing result to the LCD 63, which then displays the pH of the solution 22.

One example illustrating the two-point self-calibration of the present invention follows. In this case, the first reference value related to the pH4 buffer equals 80, the second reference value related to the pH7 buffer equals 124, and the digital value related to the solution equals 145. The sensitivity of the pH-ISFET in this case is

$$S = \frac{( pH4 - pH7 )}{3} = \frac{124 - 80}{3} = 15 ( mV \text{/} pH )$$

The sensitivity S of the pH-ISFET is obtained by the microcontroller 62, and is output to the LCD 63 as the “***” in step S40. The corresponding values of pH1˜pH13 are obtained as follows, and the relational table of digital value and the pH is created.

pH1=pH4−(pH7−pH4)

pH10=pH7=(pH7−pH4)

pH2=pH1+S

pH3=pH4−S

| pH | pH1 | pH2 | pH3 | pH4 | pH5 | pH6 | pH7 |
|---|---|---|---|---|---|---|---|
| Digital | 36 | 51 | 80 | 95 | 109 | 124 | 139 |
| value | | | | | | | |

| pH | pH8 | pH9 | pH10 | pH11 | pH12 | pH13 |
|---|---|---|---|---|---|---|
| Digital | 139 | 153 | 168 | 183 | 197 | 212 |
| value | | | | | | |

The microcontroller 62 is apprised that the digital value of 145 is between pH8 and pH9, and the value in the first decimal is obtained from

(145−pH8)×(10/S)=(145−139)×(10/15)=4

Consequently, the relative pH of digital value of 145 is 8.4, such that the value of the solution is obtained.

Finally, the measuring process of the invention is accomplished, or it goes back to step S40 if additional measurement is required.

According to the figures and the embodiments mentioned above, the present invention uses a constant voltage constant current (CVCC) circuit as the signal detect unit to improve temperature that affects the sensitivity of the pH-ISFET most, and adjusts the operational parameter of the pH-ISFET to make the voltage detected by the detector to fit in with the subsequent circuit for increasing resolution and stability. The present invention further obtains the sensitivity of the pH-ISFET by two-point calibration and estimates the pH of the solution accurately, and the present invention uses a LCD to display the pH of the solution clearly in the last stage to reduce power consumption and cost.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Thus, the scope of the appended claims should be accorded the broadest interpretation so as to encompass all such modifications and similar arrangements.

What is claimed is:

1. A portable pH detector, comprising:
a ISFET having a sensing film for immersion in a solution;
a signal detection unit to maintain a drain-source voltage and a drain-source current of the ISFET in a constant condition, and to detect the gate voltage of the ISFET as an output signal;
a gain control and level shift unit to adjust the level of the output signal as a first signal;
a A/D converter to convert the first signal into a first digital signal; and
a microprocessor coupled to the A/D converter to compare the first digital signal with a relational table to obtain the pH of the solution.

2. The portable pH detector of claim 1, wherein the microprocessor comprises:
a 8051 microcontroller coupled to the A/D converter circuit to produce a converter enable signal to enable the A/D converter circuit to convert the first signal into the first digital signal and to compare the first digital signal with the relational table to obtain the pH of the solution; and
a LCD coupled to the 8051 microcontroller to display the pH of the solution.

3. The portable pH detector of claim 2, wherein the relational table is stored in the 8051 microcontroller before testing the solution, and the relational table is created by measuring solutions with pH4 and pH7 for obtaining two corresponding digital values and by calculating corresponding digital values of pH1 to pH13 according to the two corresponding digital values of pH4 and pH7.

4. The portable pH detector of claim 1, wherein the gain control and level shift unit comprises:
an instrumentation amplifier coupled to the output signal and a reference voltage to subtract the level of the reference voltage from the level of the output signal and output a subtracted-level signal;
an operational amplifier coupled to the instrumentation amplifier to amplify the subtracted-level signal for a predetermined time as the first signal.

5. The portable pH detector of claim 4, wherein the gain control and level shift unit further comprises a potential supply circuit having:
a first resistor;
a second resistor coupled to the first resistor in series connection;
a zener diode coupled to the first resistor and the second resistor in parallel connection; and
a DC voltage source across two ends of the zener diode, wherein the DC voltage source is divided by the first resistor and the second resistor as the reference voltage output to the instrumentation amplifier.

6. The portable pH detector of claim 1, wherein the A/D converter circuit is an analog-to-digital converter (IC0804).

7. The portable pH detector of claim 3, wherein the microcontroller and the display reset when power is turned on.

8. The portable pH detector of claim 1, wherein the ISFET is one of a-WO3 gate ISFET or a-C:H gate ISFET.

9. The portable pH detector of claim 1, wherein the ISFET is a-SnO2 gate ISFET.

10. The portable pH detector of claim 1, wherein the ISFET is a-Si:H gate ISFET.

11. A method for measuring pH of a solution, compatible with a portable pH detector having a pH-ISFET with a sensing film, a signal detection unit to maintain a drain-source voltage and a drain-source current of the ISFET in a constant condition and detecting the gate voltage of the pH-ISFET, a gain control and level shift unit, a A/D converter and a microprocessor, the method comprising:
measuring a first solution with a first determined pH and a second solution with a second determined pH to obtain a first reference value and a second reference value by the pH-ISFET respectively;
creating a relational table according to the first reference value and a second reference value;
measuring a solution by the pH-ISFET to obtain a first digital value; and
comparing the first digital value and the relational table to determine the pH of the solution.

12. The method for measuring pH of a solution of claim 11, further comprising step of:
initializing the microprocessor; and
checking whether the drain-source voltage and the drain-source current provided by the signal detect unit are compatible with the pH-ISFET.

13. The method for measuring pH of a solution of claim 12, further comprising a step of adjusting the drain-source voltage and the drain-source current to compatible with the pH-ISFET by modifying the first resistor and the second resistor when the drain-source voltage and the drain-source current provided by the signal detect unit are not compatible with the pH-ISFET.

14. The method for measuring pH of a solution of claim 11, wherein steps of measuring a first solution with a first determined pH and a second solution with a second determined pH for creating the relational table comprises:
immersing the sensing film of the pH-ISFET in the first solution with the first determined pH;
measuring the gate voltage of the pH-ISFET after a first time interval;
adjusting the level of the gate voltage to an adequate level;
converting the gate voltage with adjusted level in to a first digital signal as the first reference value;
immersing the sensing film of the pH-ISFET in the second solution with the second determined pH, and measuring, adjusting, and converting the gate voltage to obtain a second digital value as the second reference value; and
creating the relational table according to the first reference value and the second reference value.

15. The method for measuring pH of a solution of claim 12, wherein the first determined time interval is 1.5 minutes.

16. The method for measuring pH of a solution of claim 13, wherein step of testing the solution to obtain the first digital value comprises:
immersing the sensing film of the pH-ISFET in the solution;
measuring the gate voltage of the pH-ISFET after a second time interval;
adjusting the level of the gate voltage to an adequate level; and
converting the gate voltage with adjusted level in to the first digital signal as the first reference value.

17. The method for measuring the pH of a solution of claim 16, wherein the second determined time interval is 1.5 minutes.

18. The method for measuring pH of a solution of claim 11, further comprising a step of displaying the pH of the solution on the microprocessor.
